(19) United States
(12) Plant Patent Application Publication
(10) Pub. No.: US 2017/0172040 P1
(43) Pub. Date: Jun. 15, 2017

(54) Cannabis plant named 'Katelyn Faith'

(50) ;

(71) Applicant: Vireo Health LLC, Minneapolis, MN (US)

(72) Inventors: Cory Carter, Minneapolis, MN (US); Kyle Kingsley, Minneapolos, MN (US)

(73) Assignee: Vireo Health LLC, Minneapolis, MN (US)

(21) Appl. No.: 14/757,239

(22) Filed: Dec. 10, 2015

(51) Int. Cl. A01H 5/12; A61K 31/352

(52) U.S. Cl. PLT/263.1

(57) ABSTRACT

The disclosure is directed to a new and distinct cultivar of Cannabis Sativa plant named ‘Katelyn Faith’, characterized by a high amount of Cannabidiol (CBD)(13.5%) and a low amount of Tetrahydrocannabinol (THC)(0.4%)

### BACKGROUND

The disclosure is directed to a new and distinct cultivar of Cannabis Sativa plant named ‘Katelyn Faith’, characterized by a high amount of Cannabidiol (CBD)(13.5%) and a low amount of Tetrahydrocannabinol (THC)(<0.4%).

### SUMMARY

Provided herein is a new and distinct Cannabis Sativa L. ssp. indica plant named Katelyn Faith, characterized by a high amount of Cannabidiol (CBD) of greater than approximately 13.5% and a low amount of Tetrahydrocannabinol (THC) of less than approximately 0.4%, as illustrated and described herein.

### BRIEF DESCRIPTION OF THE DRAWINGS

FIG. 1. illustrates a single fully grown plant of the variety;

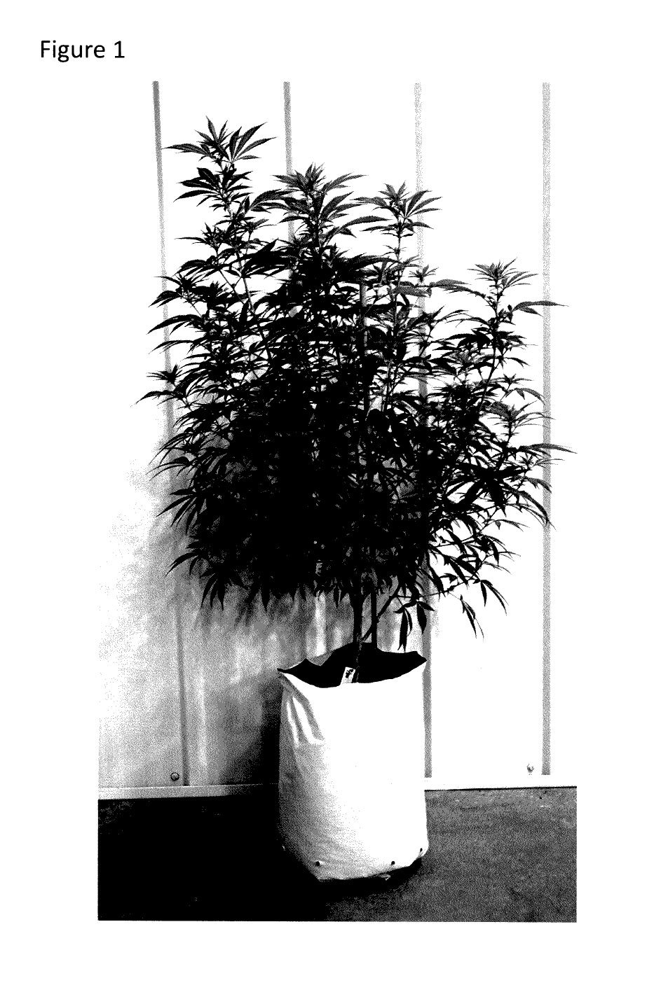

FIG. 2. illustrates a single leaf of the variety;

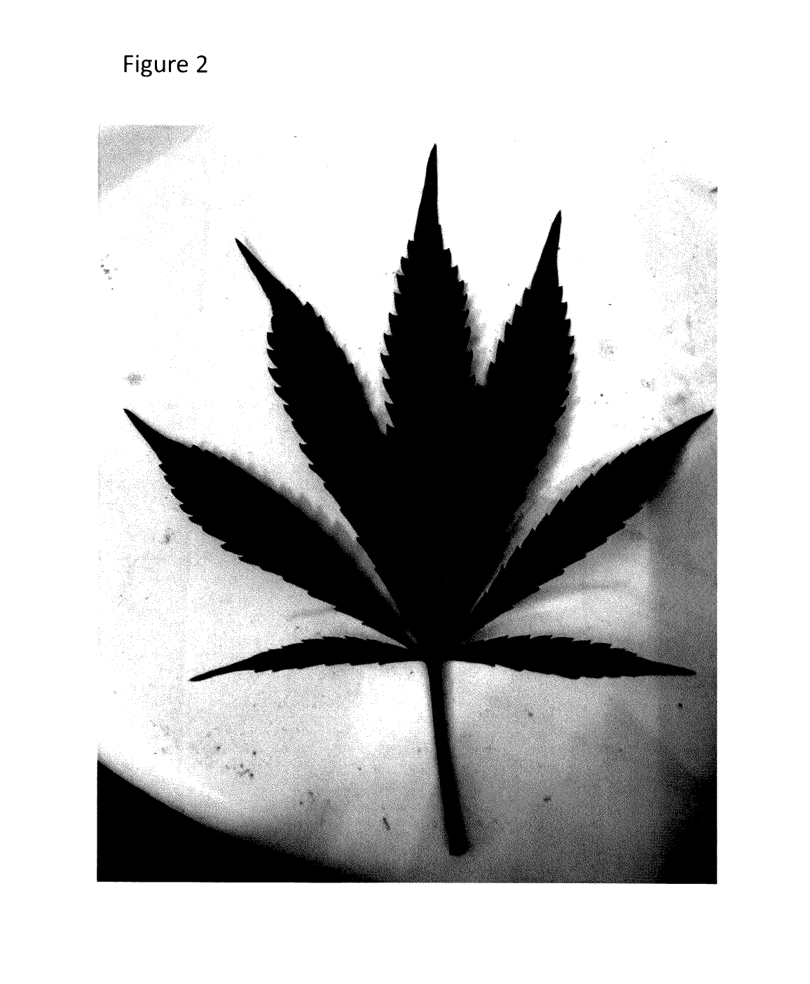

FIG. 3. illustrates a flowering plant in close up; and

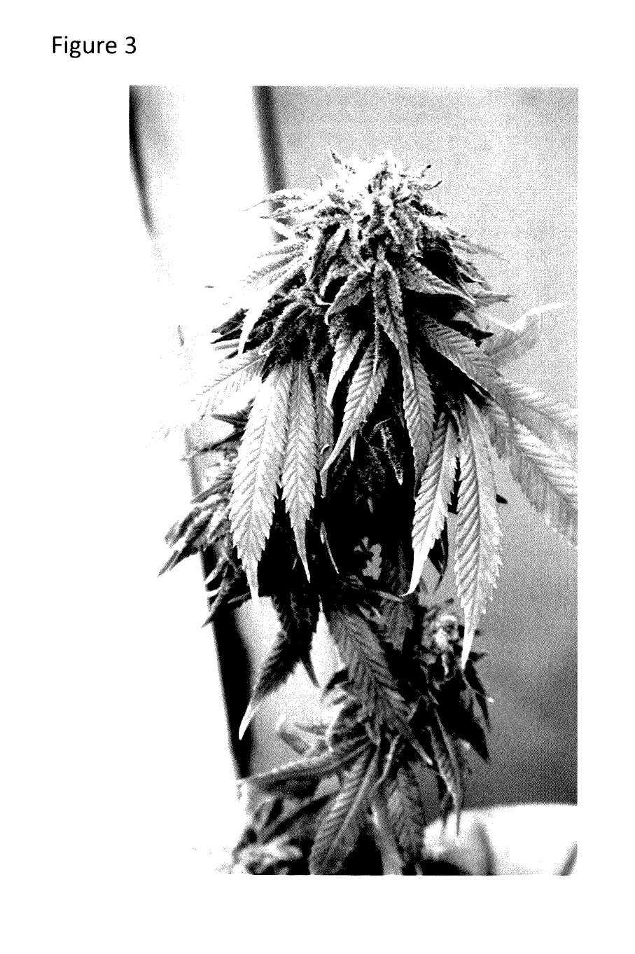

FIG. 4. illustrates a dried bud.

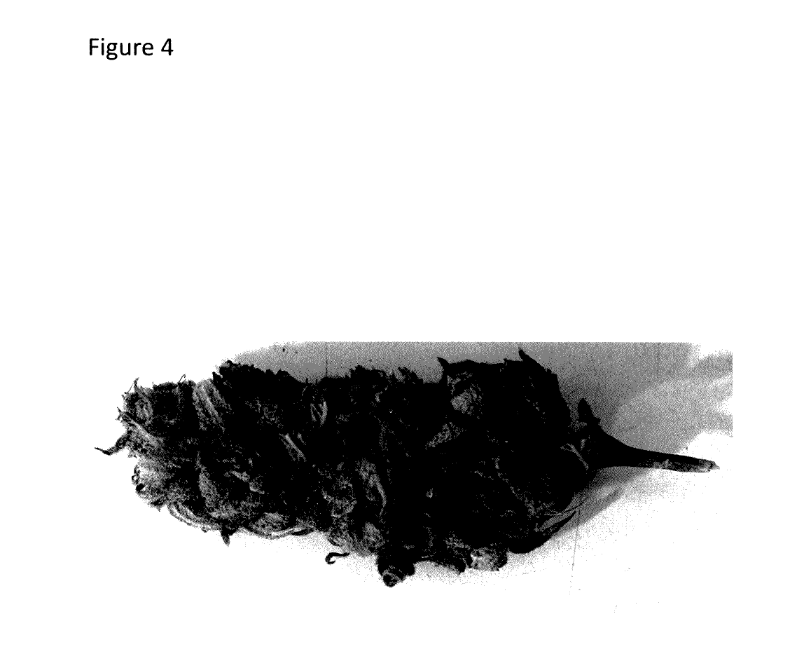

### DESCRIPTION

This invention relates to a new hybrid marijuana plant, Cannabis Sativa L. ssp indica named Katelyn Faith. The Katelyn Faith plant is a medicinal variety of marijuana notable for a high amount of Cannabidiol (CBD)(13.5%) and a very low amount of Tetrahydrocannabinol (THC)(<0.4%).

The present plant was developed over the course of 1 year through selective breeding from parents of unknown provenance in Otsego, Minn. Generally, production occurs through asexual propagation using stem cuttings. The plant may be grown indoors, such as for example, in a greenhouse or other suitable indoor growing environment under controlled conditions.

### PLANT

Exposed plant structure: The plant is an annual, dioecious plant with bush-like stature. The natural height at 6 months is 165-175 cm. A detailed list of characteristics follows:
   Botanical classification.—Cannabis Sativa L. ssp. indica.
   Parentage.—Unknown.
   Propagation.—Stem cuttings.
   Time to initiate roots in summer conditions.—10-14 days.
   Plant description.—Annual dioecious flowering shrub; multi stemmed with prolific lateral branching, medium internodal spacing; removal of the terminal bud enhances lateral branch development.
   Root description.—Medium tap root (>30 cm), fine dense lateral roots that are white in color, the roots are brownish when “potbound” (rootbound).
First year stems:
   Shape.—Round. Moderate even pubescence.
   Color.—Young stem color 144C; older stem color 144A (The Royal Horticultural Society Colour Chart, 1995 Edition.) Stem diameter at the soil line is 6.35-7.5 cm. Middle of plant average stem diameter is 2.5-3 cm. Internode spacing is 5 cm at the top half of the plant and 3.8 cm at the bottom of the plant.
Foliage description:
   Branch strength.—Medium to weak.
   Branch description.—Lateral branches off the main stem are fine and of medium strength, they contain few leaves with many bud sites extending up the branch. Branches are long and sparse.
   Leaf arrangement.—Palmately compound (digitate) with 7-9 serrated leaflets. The bottom two leaflets extend out from the petiole at approximately 180 degrees.
   Leaf texture front.—Quite supple with pliable waxy cuticle.
   Leaf texture back.—Fine white hairs are distributed on the back surface of the leaf.
   Venation pattern.—Venation of each leaf is palmately compound. Lateral venation extends off the main vein to each serrated tip. Sublateral veins extend to the notch of each serration rather than the tip.
   Color of emerging foliage (upper surface).—The color of emerging foliage falls between 144A and 144C (The Royal Horticultural Society Colour Chart, 1995 Edition.)
   Petiole length.—Average length of petiole of fan leaves is 5 cm. Petioles are very sturdy and appear a light burgundy color. The petioles are covered evenly with fine hairs.
Bud description: Dried flower buds are a dark green, smaller in stature, diffuse and airy, and coated with glandular trichomes. Fragrance is quite spicy with an earthy aroma with noticeable hints of clove.
Flower description: Inflorescence is cone shaped. The flower is devoid of any petals. Rather, it is made up of a cluster of false spikes with single flowers. These flowers are often paired and enclosed by a bracteole. Wet flower buds are bright green and covered in trichomes. Buds have many white pistils that turn a rusty red color as the plant nears maturation. Bud spike length ranges from 4-20 cm. Flower size is approximately 0.75 in diameter and approximately 1.5 cm in height. Flowering time is 8 weeks.
Peduncles: Peduncle strength is high. Texture is smooth with few hairs. Pedicels are short, with visible hairs. They are scabrid with sessile glands.
Seed production on this strain is difficult. Seed production can be induced using colloidal silver solution but even with this step male inflorescence production is marginal. Pollen generated from this procedure is then collected and used to self-cross with a non treated female plant.
This cultivar is unique and does not resemble any plants described to date.
This plant prefers hard water.
This plant, as well as isolates and extracts therefrom, has substantial medical utility. It can be used to treat spasms, Parkinson's disease, Tourette syndrome, Epilepsy, MS, Dravet's syndrome, inflammation, digestive disorders, and any other diseases or conditions for which CBD is beneficial.

| USDA Plants Growth Habit Code | FB |
|---|---|
| Vigor | 5 |
| Productivity | Good |
| Male flowering timing | 3 weeks after 12 hour induction |
| Flowering Score | 6 |
| Branches | Somewhat weak |
| THC level | 0.4 |
| CBD level | 13.5 |

A crop was grown having the following times of growth cycle operations:

| | |
|---|---|
| Date of cutting | 6/13 |
| Date transferred to growth medium | 6/23 |
| Duration of vegetative growth | 84 days |
| Duration of flowering cycle | 63 days |
| Wet flower yield (grams) | 620 grams |
| Wet leaves yield (grams) | 90 grams |
| Wet branches yield (grams) | 136 grams/sq. meter |
| Dry flower yield (grams) | 485 grams/sq. meter |
| Height of mother plants | 168 cm |
| Growth medium and size | Coarse 50:50 mix of |
| | peat and perlite |

Permits and or licenses are required for the growth of medical marijuana in certain states in the U.S. and various foreign jurisdictions.

We claim:

1. A new and distinct Cannabis Sativa L. ssp indica plant named Katelyn Faith, characterized by a high amount of Cannbidiol of greater than approximately 13.5% and a lower amount of Tetrahydrocannabinol of less than approximately 0.4%, as illustrated and described herein.
